(19) United States
(12) Patent Application Publication
(10) Pub. No.: US 2026/0041731 A1
(43) Pub. Date: Feb. 12, 2026

(54) Oyster peptide kidney-nourishing formula composition and preparation method

(71) Applicant: NANO BIOLOGY LIMITED, RENTON, WA (US)

(72) Inventors: Xinxin Lu, Ankang (CN); Likang Chen, Ankang (CN); Yukang Zhang, Ankang (CN)

(21) Appl. No.: 18/798,069

(22) Filed: Aug. 8, 2024

(51) Int. Cl. A61K 38/17; A61K 9/48; A61K 31/01; A61K 31/198; A61K 31/352; A61K 31/4415; A61K 31/525; A61K 33/04; A61K 33/30; A61K 36/31; A61K 36/42; A61K 36/71

(52) U.S. Cl. CPC A61K 38/1767; A61K 9/4825; A61K 31/01; A61K 31/198; A61K 31/352; A61K 31/4415; A61K 31/525; A61K 33/04; A61K 33/30; A61K 36/31; A61K 36/42; A61K 36/71

(57) ABSTRACT

The present application relates to composition for nourishing the kidneys and its preparation method. More specifically, the present application relates to an Oyster peptide kidney-nourishing formula composition and preparation method. The composition and preparation method are designed to address the technical issues in traditional Chinese medicine where the kidney-nourishing effects are slow to appear, treatment takes a long time, boiled medicine has a poor taste, and it's inconvenient to carry. An Oyster peptide kidney-nourishing formula composition, comprising the following medicines: oyster peptide, pumpkin seed extra, epimedium extract, maca extract, tribulus terrestris extract, eurycomanone, arginine, lycopene, VB2, VB6, selenium and zinc. The product created by this application has several functions such as increasing male serum testosterone levels, regulating blood lipids, inhibiting platelet aggregation, improving symptoms of hyperglycemia, enhancing human immunity, and promoting metabolism.

### FILD OF THE INVENTION

The present application relates to composition for nourishing the kidneys and its preparation method. More specifically, the present application relates to an Oyster peptide kidney-nourishing formula composition and preparation method.

### BACKGROUND

The kidneys are vital organs in the human body with the main function of filtering waste and excess water from the blood to produce urine. They also play a role in regulating the body's water and electrolyte balance, acid-base balance, and endocrine balance. The health of the kidneys has a direct impact on the overall health of the body.

Nourishing the kidneys is an important concept in traditional Chinese medicine. According to this theory, the kidneys are considered the innate foundation of the human body, responsible for storing essence, regulating water, controlling growth and development, among other functions. When there is a deficiency in the kidneys, individuals may experience symptoms such as lower back and knee soreness, dizziness, nocturia (frequent urination at night), sexual dysfunction, premature ejaculation, dry mouth and throat, and sensitivity to cold.

Currently, traditional Chinese medicine primarily employs methods such as Chinese medicine conditioning, acupuncture, moxibustion, and massage to address symptoms associated with kidney deficiency. However, these treatments are slow to show results, require a lengthy treatment period, and the consumption of boiled Chinese medicine is inconvenient due to its unpalatable taste.

### SUMMARY

The present application overcomes the limitations of existing technology by providing an Oyster peptide kidney-nourishing formula composition and preparation method. The composition and preparation method are designed to address the technical issues related to traditional Chinese medicine. These issues include the slow manifestation of the kidney-nourishing effect, the prolonged treatment duration, the poor taste of boiled traditional Chinese medicine, and the inconvenience associated with it.

An Oyster peptide kidney-nourishing formula composition, comprising the following medicines: oyster peptide, pumpkin seed extra, epimedium extract, maca extract, tribulus terrestris extract, eurycomanone, arginine, lycopene, VB2, VB6, selenium and zinc.

In an embodiment, the ratio is as follows by weight:
oyster peptide 50-300 mg
pumpkin seed extra 50-300 mg
epimedium extract 10-200 mg
maca extract 10-200 mg
tribulus terrestris extract 10-100 mg
eurycomanone 10-100 mg
arginine 10-300 mg
lycopene 10-100 mg
VB2 0.1-10 mg
VB6 0.1-10 mg
selenium 0.1-10 mg
zinc 0.1-10 mg

[symbol]

An Oyster peptide kidney-nourishing formula composition and preparation method, comprising the Oyster peptide kidney-nourishing formula composition of claim 1 or 2, the steps are as follows:
Step 1: weighing, after sieving the medicines through a 50-60 mesh, oyster peptide, pumpkin seed extract, epimedium extract, maca extract, tribulus extract, tongkat ali ketone, arginine, lycopene, VB2, VB6, selenium, and zinc are weighed according to the specified ratio;
Step 2: mixing, the medicines mentioned above are combined in a mixer and mixed slowly for 20 to 30 minutes to create a composition;
Step 3: detecting, the prepared mixture is put into the capsule shell, and the shell is sealed with a cover to prevent any leaks; once the capsule is assembled, it is weighed, and any capsules that are overweight or underweight are taken out;
Step 4: packing, place the prepared capsules into the outer packaging with positioning grooves one by one, seal the upper end of the outer packaging, and mark the capsule's ingredients, use, production date, shelf life, etc. on the seal.
   Wherein the capsules in step 3 are made of either gelatin or vegetable capsules.
   Wherein the speed of the mixer in step 2 is 10 to 16 rpm; the outside of the mixer in step 2 has a shaking structure.
   Wherein the packaging specification of step 4 is 300 mg/tablet*12 tablets.
   Wherein step 3 utilizes automatic or semi-automatic capsule filling machines for filling capsules.

### BENEFICIAL EFFECTS OF THE INVENTION

The product created by this application has several functions, including increasing male serum testosterone levels, regulating blood lipids, inhibiting platelet aggregation, improving hyperglycemia symptoms, enhancing human immunity, and promoting metabolism. It also has some effects in terms of anti-cancer properties and preventing the spread of cancer cells. Furthermore, when made into capsules, it becomes easy to carry and take with accurate dosage, making it suitable for the fast-paced lifestyle of modern people. This is more conducive to treatment adherence and avoids the issues of bitterness and difficulty in swallowing that come with traditional Chinese medicine decoctions.

While the technology is susceptible to various modifications and alternative forms, specifics thereof have been shown by way of example and drawings, and will be described in detail. It should be understood, however, that the application is not limited to the particular embodiments described. On the contrary, the application is to cover modifications, equivalents, and alternatives falling within the spirit and scope of the technology.

### DETAILED DESCRIPTION OF EMBODIMENTS

The embodiments of the present technology described herein are not intended to be exhaustive or to limit the technology to the precise forms disclosed in the following detailed description. Rather, the embodiments are chosen and described so that others skilled in the art can appreciate and understand the principles and practices of the present technology.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

### EMBODIMENT 1

In this embodiment, an Oyster peptide kidney-nourishing formula composition, comprising the following medicines: oyster peptide, pumpkin seed extra, epimedium extract, maca extract, tribulus terrestris extract, eurycomanone, arginine, lycopene, VB2, VB6, selenium and zinc.

Wherein, the ratio is as follows by weight:
oyster peptide 150 mg
pumpkin seed extra 70 mg
epimedium extract 30 mg
maca extract 30 mg
tribulus terrestris extract 20 mg
eurycomanone 10 mg
arginine 15 mg
lycopene 15 mg
VB2 1 mg
VB6 2 mg
selenium 1 mg
zinc 1.5mg

[symbol]

An Oyster peptide kidney-nourishing formula composition and preparation method, comprising the Oyster peptide kidney-nourishing formula composition of above, the steps are as follows:
Step 1: weighing, after sieving the medicines through a 50 mesh, oyster peptide, pumpkin seed extract, epimedium extract, maca extract, tribulus extract, tongkat ali ketone, arginine, lycopene, VB2, VB6, selenium, and zinc are weighed according to the specified ratio;
   S2 [symbol] 20 min [symbol]
Step 3: detecting, the prepared mixture is put into the capsule shell, and the shell is sealed with a cover to prevent any leaks; once the capsule is assembled, it is weighed, and any capsules that are overweight or underweight are taken out;
Step 4: packing, place the prepared capsules into the outer packaging with positioning grooves one by one, seal the upper end of the outer packaging, and mark the capsule's ingredients, use, production date, shelf life, etc. on the seal.

The Oyster peptide kidney-nourishing formula composition in embodiment 1 has a calming effect, reduces medicine-induced irritation to the body, and effectively treats symptoms of kidney deficiency.

### EMBODIMENT 2

In this embodiment, an Oyster peptide kidney-nourishing formula composition, comprising the following medicines: oyster peptide, pumpkin seed extra, epimedium extract, maca extract, tribulus terrestris extract, eurycomanone, arginine, lycopene, VB2, VB6, selenium and zinc.

Wherein, the ratio is as follows by weight:
oyster peptide 200 mg
pumpkin seed extra 100 mg
epimedium extract 85 mg
maca extract 45 mg
tribulus terrestris extract 35 mg
eurycomanone 30 mg
arginine 100 mg
lycopene 15 mg
VB2 1.5 mg
VB6 3 mg
selenium 3 mg
zinc 2.5 mg

[symbol]

An Oyster peptide kidney-nourishing formula composition and preparation method, comprising the Oyster peptide kidney-nourishing formula composition of above, the steps are as follows:
Step 1: weighing, after sieving the medicines through a 60 mesh, oyster peptide, pumpkin seed extract, epimedium extract, maca extract, tribulus extract, tongkat ali ketone, arginine, lycopene, VB2, VB6, selenium, and zinc are weighed according to the specified ratio;
Step 2: mixing, the medicines mentioned above are combined in a mixer and mixed slowly for 20 to 30 minutes to create a composition;
Step 3: detecting, the prepared mixture is put into the capsule shell, and the shell is sealed with a cover to prevent any leaks; once the capsule is assembled, it is weighed, and any capsules that are overweight or underweight are taken out;
Step 4: packing, place the prepared capsules into the outer packaging with positioning grooves one by one, seal the upper end of the outer packaging, and mark the capsule's ingredients, use, production date, shelf life, etc. on the seal.

The Oyster peptide kidney-nourishing formula composition in embodiment 2 could rapidly and effectively demonstrate a therapeutic effect on the disease.

1. An Oyster peptide kidney-nourishing formula composition, comprising the following medicines: oyster peptide, pumpkin seed extra, epimedium extract, maca extract, tribulus terrestris extract, eurycomanone, arginine, lycopene, VB2, VB6, selenium and zinc.

2. The Oyster peptide kidney-nourishing formula composition of claim 1, comprising by weight, the ratio is as follows:
oyster peptide 50-300 mg
pumpkin seed extra 50-300 mg
epimedium extract 10-200 mg
maca extract 10-200 mg
tribulus terrestris extract 10-100 mg
eurycomanone 10-100 mg
arginine 10-300 mg
lycopene 10-100 mg
VB2 0.1-10 mg
VB6 0.1-10 mg
selenium 0.1-10 mg
zinc 0.1-10 mg

[symbol]

3. An Oyster peptide kidney-nourishing formula composition and preparation method, comprising the Oyster peptide kidney-nourishing formula composition of claim 1, the steps are as follows:
step 1: weighing, after sieving the medicines through a 50-60 mesh, oyster peptide, pumpkin seed extract, epimedium extract, maca extract, tribulus extract, tongkat ali ketone, arginine, lycopene, VB2, VB6, selenium, and zinc are weighed according to the specified ratio;
step 2: mixing, the medicines mentioned above are combined in a mixer and mixed slowly for 20 to 30 minutes to create a composition;
step 3: detecting, the prepared mixture is put into the capsule shell, and the shell is sealed with a cover to prevent any leaks; once the capsule is assembled, it is weighed, and any capsules that are overweight or underweight are taken out;
step 4: packing, place the prepared capsules into the outer packaging with positioning grooves one by one, seal the upper end of the outer packaging, and mark the capsule's ingredients, use, production date, shelf life, etc. on the seal.

4. The Oyster peptide kidney-nourishing formula composition and preparation method of claim 3, comprising, the capsules in step 3 are made of either gelatin or vegetable capsules.

5. The Oyster peptide kidney-nourishing formula composition and preparation method of claim 3, comprising, the speed of the mixer in step 2 is 10 to 16 rpm; the outside of the mixer in step 2 has a shaking structure.

6. The Oyster peptide kidney-nourishing formula composition and preparation method of claim 3, comprising, the packaging specification of step 4 is 300 mg/tablet*12 tablets.

7. The Oyster peptide kidney-nourishing formula composition and preparation method of claim 3, comprising, step 3 utilizes automatic or semi-automatic capsule filling machines for filling capsules.
